(19) United States
(12) Patent Application Publication
(10) Pub. No.: US 2017/0347862 A1
(43) Pub. Date: Dec. 7, 2017

(54) ENDOSCOPE CONNECTOR

(71) Applicant: OLYMPUS CORPORATION, Tokyo (JP)

(72) Inventor: Kahori YASUNAGA, Hino-shi (JP)

(73) Assignee: OLYMPUS CORPORATION, Tokyo (JP)

(21) Appl. No.: 15/686,284

(22) Filed: Aug. 25, 2017

Related U.S. Application Data
(63) Continuation of application No. PCT/JP2016/060084, filed on Mar. 29, 2016.

(30) Foreign Application Priority Data
Jun. 2, 2015 (JP) 2015-112481

(51) Int. Cl. A61B 1/00; A61B 1/07

(52) U.S. Cl. CPC A61B 1/00128; A61B 1/07; A61B 1/00126; A61B 1/00114; A61B 1/00119; A61B 1/00117; A61B 1/00124

(57) ABSTRACT

An endoscope connector for connecting an endoscope to an external apparatus, includes: an exterior member including a first opening and a second opening on end part side, the first opening and the second opening respectively exposing, to an outside, predetermined members of the endoscope to be connected to the external apparatus; and a supporting member that is disposed inside the exterior member and holds the predetermined members, in which the supporting member includes an insertion hole that faces a region on the end part side of the exterior member, the region being different from the first opening and the second opening.

### CROSS REFERENCE TO RELATED APPLICATION

This application is a continuation application of PCT/JP2016/060084 filed on Mar. 29, 2016 and claims benefit of Japanese Application No. 2015-112481 filed in Japan on Jun. 2, 2015, the entire contents of which are incorporated herein by this reference.

### BACKGROUND OF THE INVENTION

### 1. Field of the Invention

The present invention relates to an endoscope connector of an endoscope attached to an external apparatus.

### 2. Description of the Related Art

In recent years, endoscopes have been widely used in a medical field. The endoscopes used in the medical field allow for observation of organs in a body cavity through insertion of an elongated insertion portion into the body cavity, and various kinds of treatment using treatment instruments that are inserted into an insertion channel for the treatment instruments, included in the endoscopes as necessary.

In addition, the configuration in which an endoscope is connected to an external apparatus by attaching an endoscope connector provided on an extended end of a universal cord to, for example, a light source apparatus, is well known.

More specifically, the configuration in which the endoscope connector is attached to a connector receiver provided on the light source apparatus, and a light guide pipe sleeve and an air feeding pipe sleeve serving as predetermined members provided on the endoscope connector are fitted into and connected to holes provided on the connector receiver, is well-known.

Note that the endoscope connector includes a supporting member serving as an internal member. The internal member holds a light guide pipe sleeve and an air feeding pipe sleeve when the light guide pipe sleeve is inserted into a light guide pipe sleeve insertion hole and the air feeding pipe sleeve is inserted into an air feeding pipe sleeve insertion hole, respectively.

In addition, the endoscope connector includes an exterior member in which the supporting member is provided. The exterior member includes a first opening and a second opening on, for example, an end surface part on end part side to be attached to the light source apparatus. The first opening exposes the light guide pipe sleeve, namely, the light guide pipe sleeve insertion hole to the outside, and the second opening exposes the air feeding pipe sleeve, namely, the air feeding pipe sleeve insertion hole to the outside.

Here, the light guide pipe sleeve and the air feeding pipe sleeve are disposed close to each other in a radial direction of the endoscope connector due to downsizing of the endoscope connector. In other words, the first opening and the second opening are disposed close to each other in the radial direction.

Japanese Patent No. 5231691 discloses a configuration of an endoscope connector in which a plate-like pipe sleeve holding member is bonded and fixed to an end surface part of an exterior member of the endoscope connector. The pipe sleeve holding member is provided separately from the exterior member and reinforces a fragile part in a region between a first opening and a second opening.

### SUMMARY OF THE INVENTION

An endoscope connector according to an aspect of the present invention is for connecting an endoscope to an external apparatus, the endoscope connector including: an exterior member including a first opening and a second opening on end part side, the first opening and the second opening respectively exposing, to an outside, predetermined members of the endoscope to be connected to the external apparatus; and an internal member that is disposed inside the exterior member and holds the predetermined members, in which the internal member includes an insertion hole that faces a region on the end part side of the exterior member, the region being different from the first opening and the second opening.

### BRIEF DESCRIPTION OF THE DRAWINGS

FIG. 1 is a perspective view illustrating an endoscope that includes an endoscope connector according to a first embodiment;

FIG. 2 is a perspective view illustrating the endoscope connector of FIG. 1 in an enlarged manner;

FIG. 3 is a perspective view of a connector connection system illustrating, together with a video processor, a state in which the endoscope connector of FIG. 2 is attached to a light source apparatus;

FIG. 4 is an enlarged perspective view illustrating end part side of the endoscope connector of FIG. 2 without a light guide pipe sleeve and an air feeding pipe sleeve;

FIG. 5 is a cross-sectional diagram of the endoscope connector along line V-V in FIG. 4;

FIG. 6 is a plan view of the end part side of the endoscope connector of FIG. 5 as viewed from a VI direction in FIG. 5;

FIG. 7 is a perspective view illustrating an internal member of FIG. 5 in an enlarged manner;

FIG. 8 is a cross-sectional diagram of the internal member along line VIII-VIII in FIG. 7;

FIG. 9 is a cross-sectional diagram of the internal member of FIG. 8 as viewed from an IX direction in FIG. 8;

FIG. 10 is a plan view of the internal member of FIG. 9 as viewed from an X direction in FIG. 9;

FIG. 11 is a perspective view illustrating, together with a sprue and runners, an exterior member on the end part side of the endoscope connector of FIG. 4;

FIG. 12 is a cross-sectional diagram of an internal member of an endoscope connector according to a second embodiment; and

FIG. 13 is a cross-sectional diagram illustrating a modification in which an inner peripheral surface of an insertion hole of a supporting member of FIG. 12 is formed in a tapered shape.

### DETAILED DESCRIPTION OF THE PREFERRED EMBODIMENTS

Some embodiments of the present invention are described below with reference to drawings. Note that the drawings are schematic illustration, and relationship between a thickness and a width of each member, a thickness ratio of members, and the like are different from actual relationship, thickness ratio, and the like. Ratios and other relationships may also differ from one drawing to another as a matter of course.

### First Embodiment

An embodiment of the present invention is described below with reference to drawings.

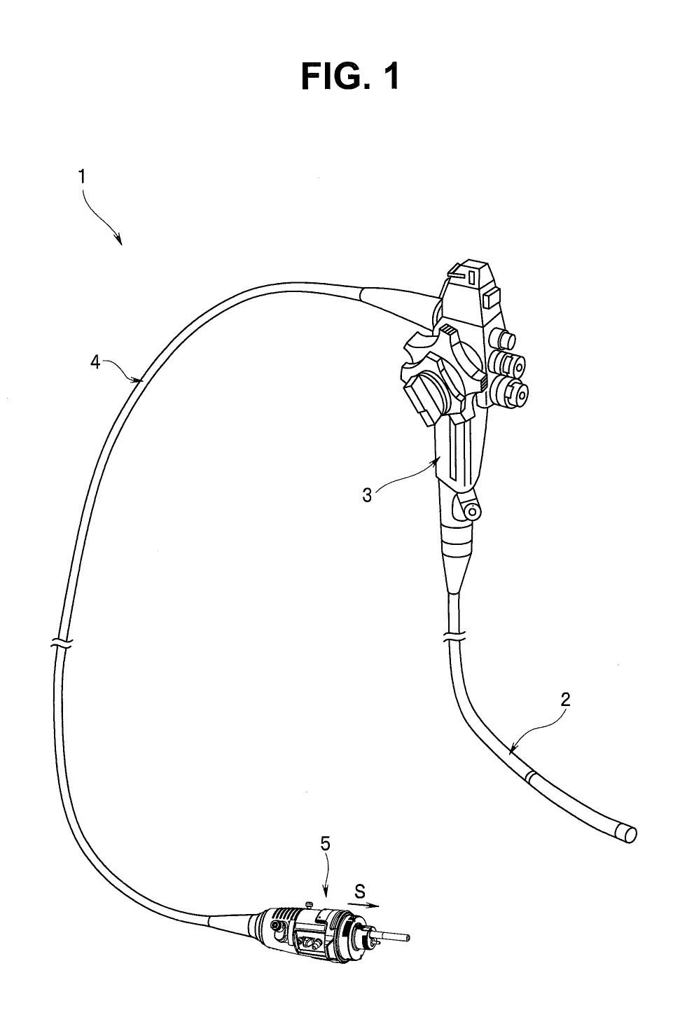

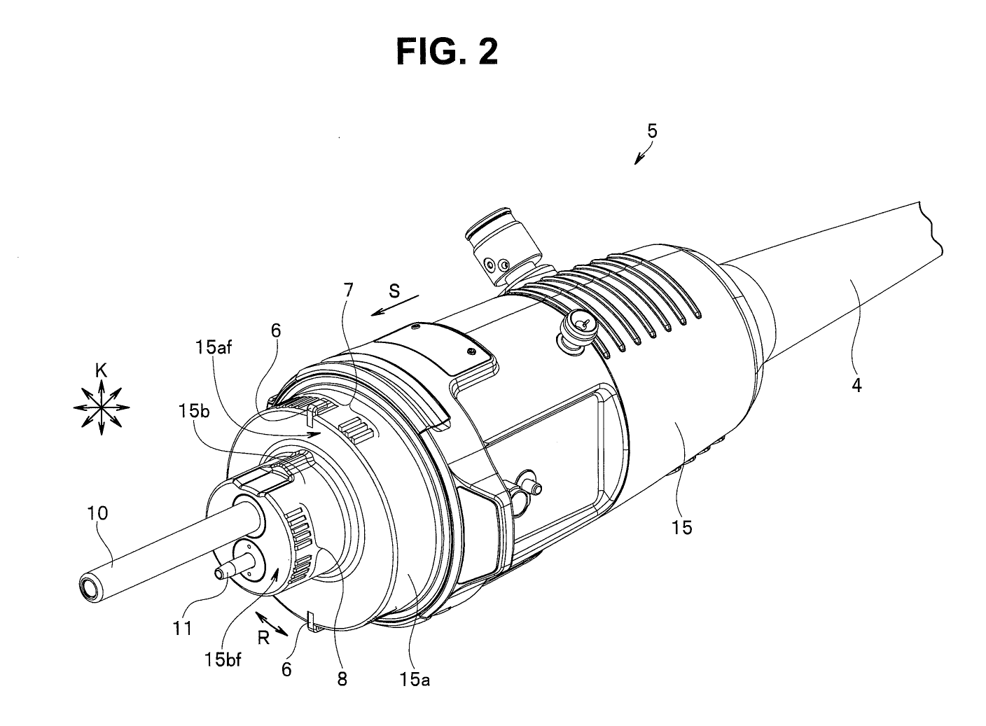

FIG. 1 is a perspective view illustrating an endoscope that includes an endoscope connector according to the present embodiment, and FIG. 2 is a perspective view illustrating the endoscope connector of FIG. 1 in an enlarged manner.

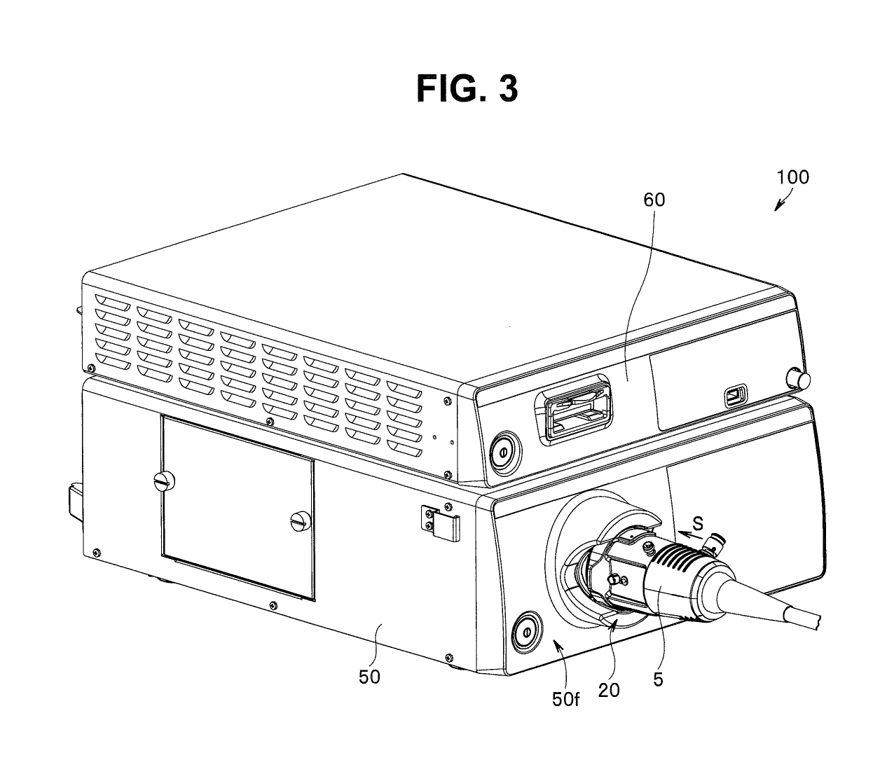

In addition, FIG. 3 is a perspective view of a connector connection system illustrating, together with a video processor, a state in which the endoscope connector of FIG. 2 is attached to a light source apparatus.

As illustrated in FIG. 1 and FIG. 3, a connector connection system 100 includes, as a main part, a connector receiver 20 and an endoscope connector 5 of an endoscope 1. The connector receiver 20 is provided on a front surface 50f of a light source apparatus 50 that is an external apparatus supplying light to the endoscope 1. The endoscope connector 5 is detachable to the connector receiver 20.

As illustrated in FIG. 1, the endoscope connector 5 is provided at an extended end of a universal cord 4 that is extended from an operation section 3 provided on proximal end side of an insertion portion 2 in the endoscope 1. The insertion portion 2 is inserted into a subject.

In addition, as illustrated in FIG. 3, a video processor 60 that performs operation control of the endoscope 1 is provided on, for example, a top surface of the light source apparatus 50. Note that the video processor 60 is electrically connected to the light source apparatus 50 through an unillustrated connection cable.

As illustrated in FIG. 2, the endoscope connector 5 includes a roughly columnar exterior member 15 that is made of, for example, a resin.

Further, end part side of the exterior member 15 is attached to parts other than the end part side of the exterior member 15. The end part side of the exterior member 15 is distal end side in a direction S in which the endoscope connector 5 is fitted into the connector receiver 20 (hereinafter, simply referred to as a fitting direction).

Moreover, end part side of the endoscope connector 5 includes a first fitting part 15a and a second fitting part 15b. The first fitting part 15a has an outer diameter smaller than an outer diameter of a part other than the end part side in a radial direction K. The second fitting part 15b projects forward from a distal end surface 15af of the first fitting part 15a in the fitting direction S and has an outer diameter smaller than the outer diameter of the first fitting part 15a in the radial direction K.

In other words, the end part side of the endoscope connector 5 is formed in a two-stage shape including the first fitting part 15a and the second fitting part 15b.

The first fitting part 15a is fitted into an unillustrated outer receptacle of the connector receiver 20 when the endoscope connector 5 is attached to the connector receiver 20. In addition, the second fitting part 15b is fitted to an unillustrated inner receptacle of the connector receiver 20 when the endoscope connector 5 is attached to the connector receiver 20.

A plurality of electrical contacts 7 are so provided on an outer peripheral surface of the first fitting part 15a through, for example, insert molding, as to be arranged along a circumferential direction R of the endoscope connector 5. In addition, a plurality of electrical contacts 8 are so provided on an outer peripheral surface of the second fitting part 15b through, for example, insert molding, as to be arranged along the circumferential direction R.

The electrical contacts 7 are electrically connected to unillustrated electrical contacts that are provided on an inner peripheral surface of the outer receptacle when the endoscope connector 5 is attached to the connector receiver 20. Moreover, the electrical contacts 8 are electrically connected to unillustrated electrical contacts that are provided on an inner peripheral surface of the inner receptacle when the endoscope connector 5 is attached to the connector receiver 20. This causes the endoscope connector 5 to be electrically connected to the light source apparatus 50.

Further, key parts 6 are provided at respective positions that face each other in the radial direction K on the distal end side of the first fitting part 15a in the fitting direction S.

Each of the key part 6 has a function of being fitted into an unillustrated keyway of the connector receiver 20 to prevent rotation of the endoscope connector 5 with respect to the connector receiver 20 in the circumferential direction R when the endoscope connector 5 is attached to the connector receiver 20.

Furthermore, a light guide pipe sleeve 10 and an air feeding pipe sleeve 11 are extended forward in the fitting direction S from a distal end surface 15bf that is an end surface part of the second fitting part 15b. The light guide pipe sleeve 10 is a predetermined member made of, for example, a metal. The air feeding pipe sleeve 11 is a predetermined member made of, for example, a metal.

The light guide pipe sleeve 10 is fitted into a hole of an unillustrated light guide socket provided inside the connector receiver 20 when the endoscope connector 5 is attached to the connector receiver 20.

In addition, the air feeding pipe sleeve 11 is fitted into a hole of an unillustrated air feeding pipe sleeve protection socket provided inside the connector receiver 20 when the endoscope connector 5 is attached to the connector receiver 20.

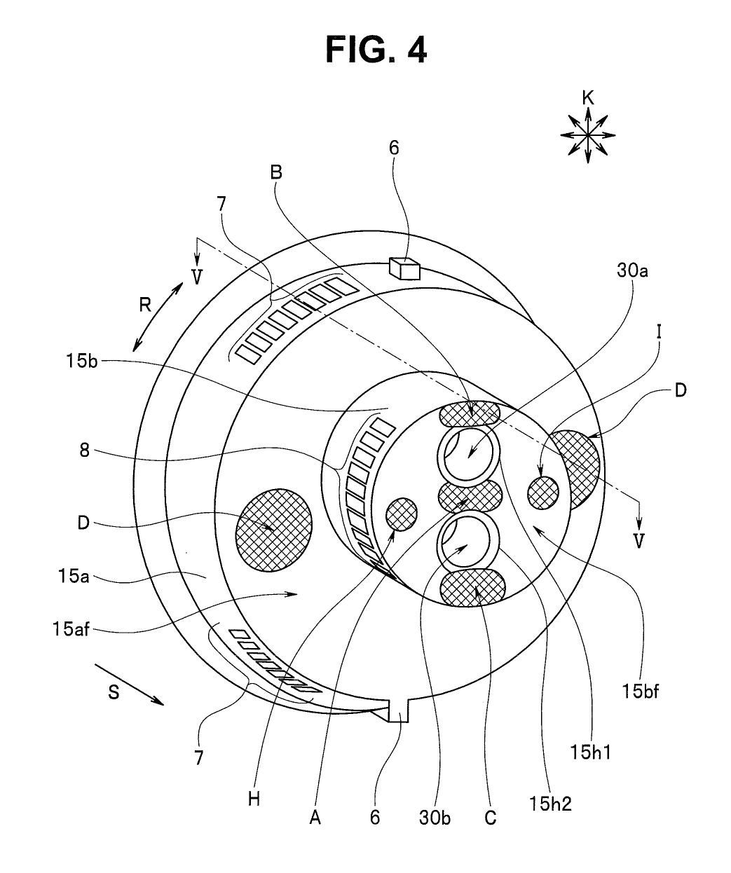

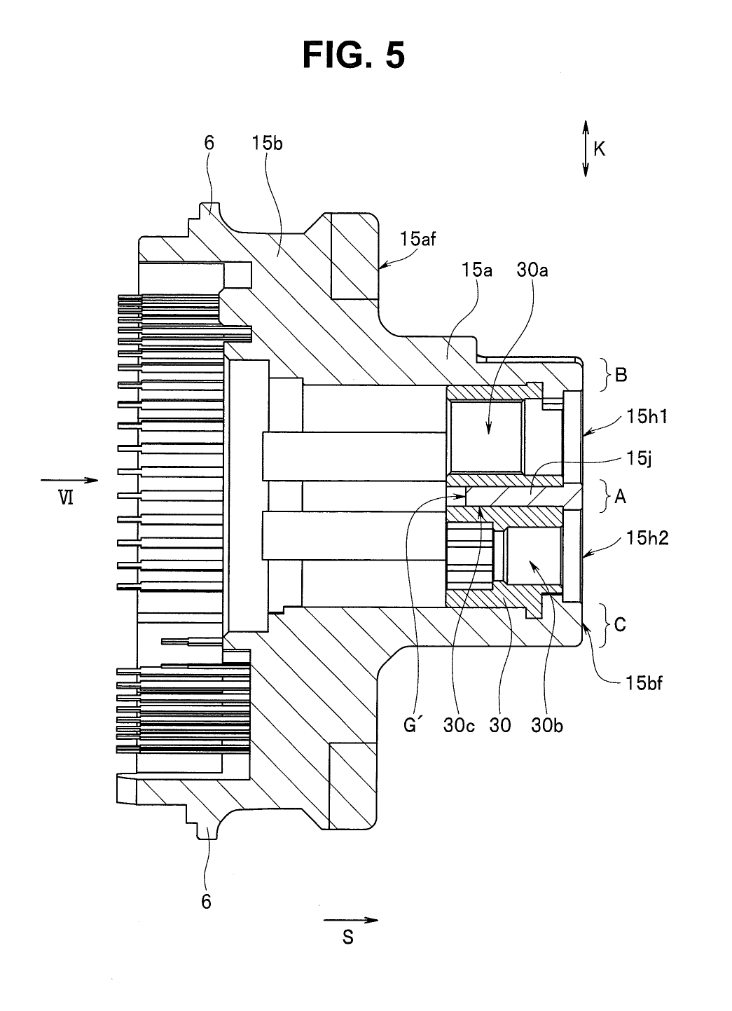

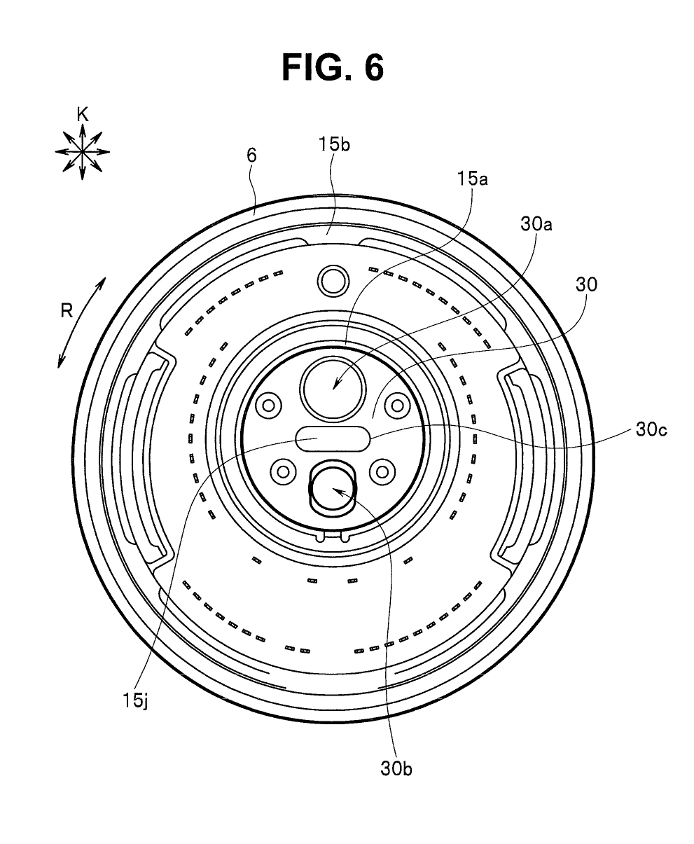

Next, a configuration of the end part side of the endoscope connector 5 is illustrated with reference to FIG. 4 to FIG. 11. FIG. 4 is an enlarged perspective view illustrating the end part side of the endoscope connector of FIG. 2 without the light guide pipe sleeve and the air feeding pipe sleeve. FIG. 5 is a cross-sectional diagram of the endoscope connector along line V-V in FIG. 4. FIG. 6 is a plan view of the end part side of the endoscope connector of FIG. 5 as viewed in a VI direction in FIG. 5.

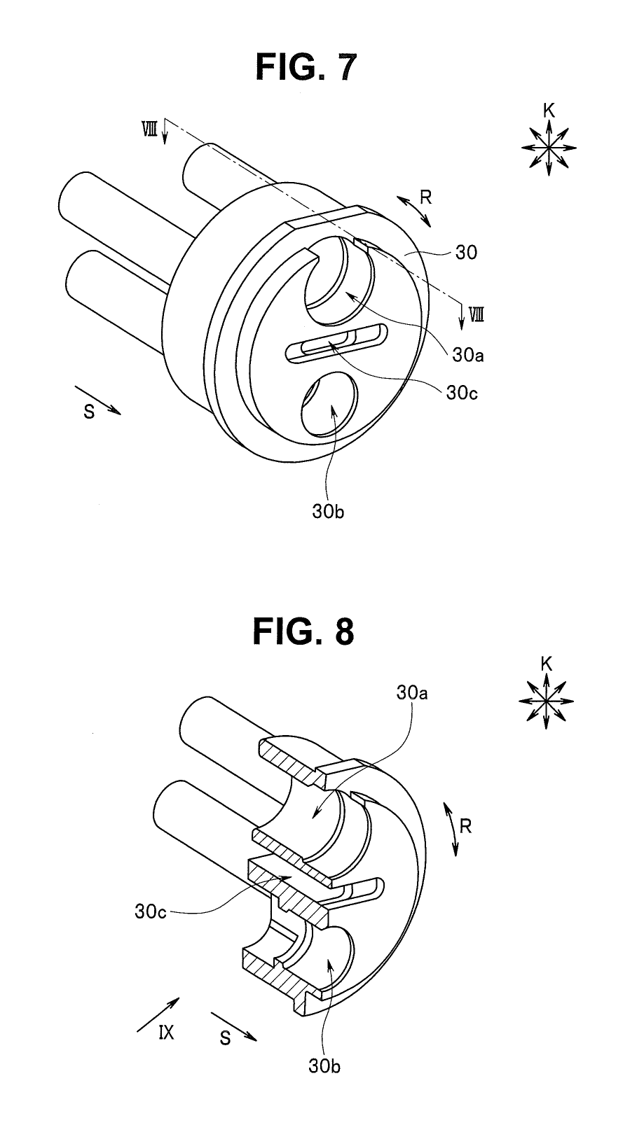

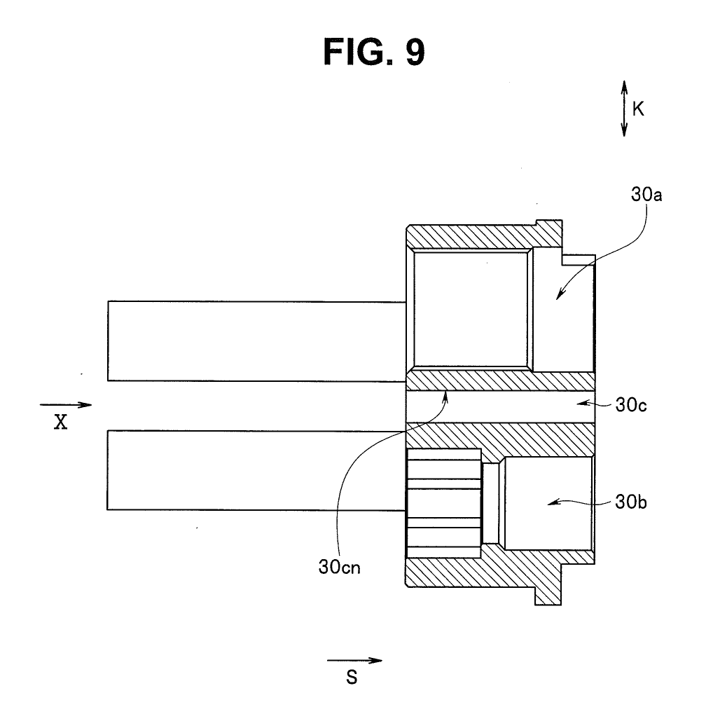

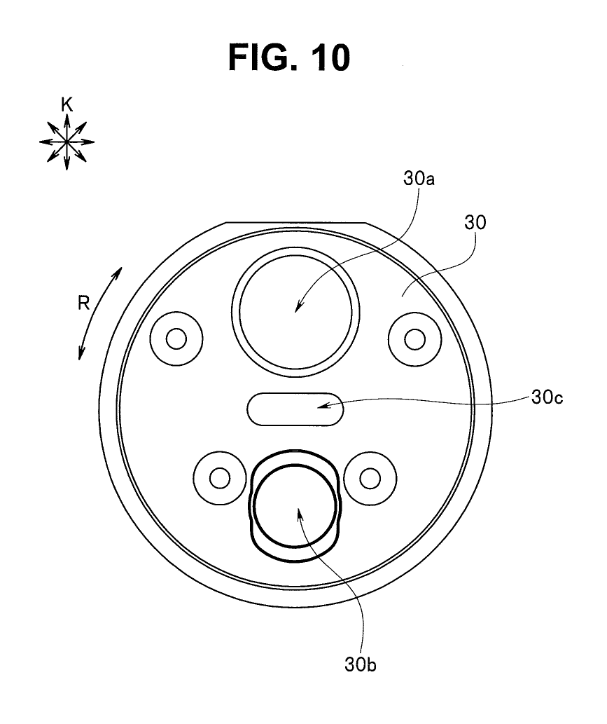

Further, FIG. 7 is a perspective view illustrating an internal member of FIG. 5 in an enlarged manner. FIG. 8 is a cross-sectional diagram of the internal member along line VIII-VIII in FIG. 7. FIG. 9 is a cross-sectional diagram of the internal member of FIG. 8 as viewed from an IX direction in FIG. 8. FIG. 10 is a plan view of the internal member of FIG. 9 as viewed from an X direction in FIG. 9.

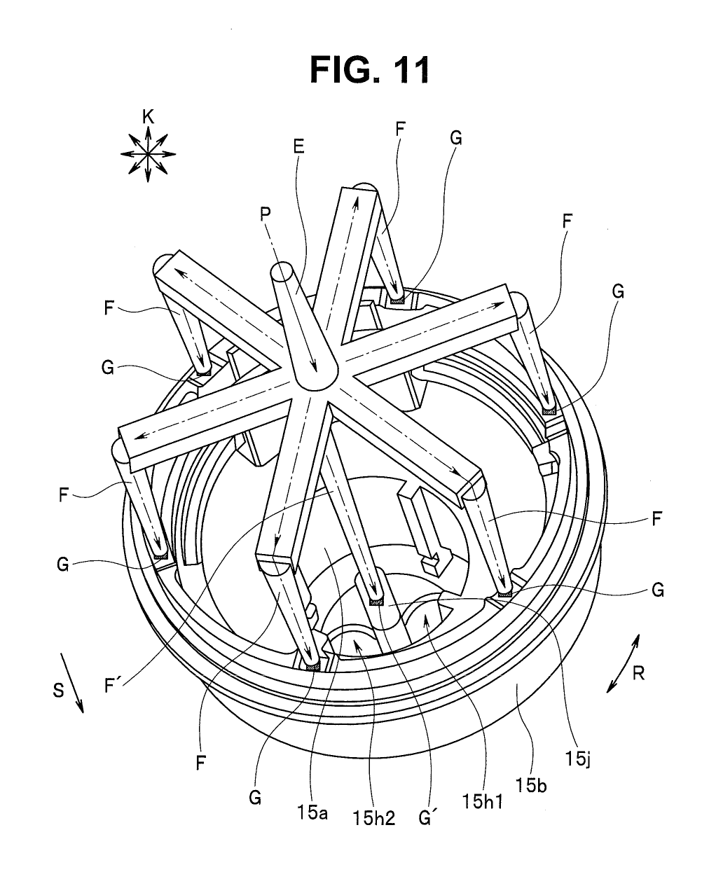

Furthermore, FIG. 11 is a perspective view illustrating, together with a sprue and runners, an exterior member on the end part side of the endoscope connector of FIG. 4.

As illustrated in FIG. 4 to FIG. 6, a supporting member 30 that holds the light guide pipe sleeve 10 and the air feeding pipe sleeve 11 is provided inside the exterior member 15 on the end part side of the endoscope connector 5, more specifically, inside the exterior member 15 of the first fitting part 15a and the second fitting part 15b. The supporting member 30 is an internal member made of, for example, a metal.

As illustrated in FIG. 5 to FIG. 10, the supporting member 30 includes a light guide pipe sleeve insertion hole 30a into which the light guide pipe sleeve 10 is inserted. The light guide pipe sleeve insertion hole 30a penetrates through the supporting member 30 in the fitting direction S so as to face the distal end surface 15bf.

In addition, the supporting member 30 includes an air feeding pipe sleeve insertion hole 30b into which the air feeding pipe sleeve 11 is inserted. The air feeding pipe sleeve insertion hole 30b penetrates through the supporting member 30 in the fitting direction S so as to face the distal end surface 15bf.

Moreover, the supporting member 30 includes an insertion hole 30c that is filled with a portion of the exterior member 15. The insertion hole 30c penetrates through the supporting member 30 in the fitting direction S so as to face a region that is different from a first opening 15h1 and a second opening 15h2 (detail of the openings is described later) of the distal end surface 15bf.

Further, as illustrated in FIG. 4 and FIG. 5, the first opening 15h1 and the second opening 15h2 are provided close to each other in the radial direction K on the distal end surface 15bf of the exterior member 15 of the second fitting part 15b. The first opening 15h1 and the second opening 15h2 respectively expose the light guide pipe sleeve 10 and the air feeding pipe sleeve 11 to the outside of the exterior member 15 as illustrated in FIG. 1 and FIG. 2.

Note that the first opening 15h1 communicates with the light guide pipe sleeve insertion hole 30a, and the second opening 15h2 communicates with the air feeding pipe sleeve insertion hole 30b.

Further, as illustrated in FIG. 5, the distal end surface 15bf includes a filling part 15j as a portion of the exterior member 15. The filling part 15j projects from the distal end surface 15bf toward the insertion hole 30c and is provided integrally with the distal end surface 15bf.

Note that a length of the filling part 15j in the fitting direction S is varied depending on a length of the insertion hole 30c in the fitting direction S. When the insertion hole 30c has an extremely short length, the filling part 15j may not be provided.

In addition, as illustrated in FIG. 4, the distal end surface 15bf includes a first region A, a second region B, and a third region C. The first region A is provided between the first opening 15h1 and the second opening 15h2 in the radial direction K. The second region B faces the first region A with the first opening 15h1 in between in the radial direction K. The third region C faces the first region A with the second opening 15h2 in between in the radial direction K.

Here, as illustrated in FIG. 5 to FIG. 10, the insertion hole 30c provided in the supporting member 30 is so provided as to face a region that is different from the second region B and the third region C, of the distal end surface 15bf. More specifically, the insertion hole 30c is so provided as to face the first region A.

Injection molding in which a resin P is injected into an unillustrated mold that molds the exterior member 15 of the first fitting part 15a and the second fitting part 15b is performed while the supporting member 30 is set in the mold. As a result, the supporting member 30 is inserted into the end part side and the entire end part side of the exterior member 15 having the above-described configuration is integrally formed.

Note that, when the resin P is injected into the mold, the resin P is injected into the mold through, for example, a sprue E, six runners F, six gates G, a runner F′, and a gate G′ as illustrated in FIG. 11, in order to be uniformly injected into the mold. The sprue E is an injection port of the resin P into the mold. The runners F are branched from the sprue E and are disposed with equivalent intervals in the circumferential direction R to face respective sites that are proximal ends, in the fitting direction S, on an outer periphery of exterior member of the first fitting part 15a after the molding. The six gates G are injection ports of the resin P to the first fitting part 15a and the second fitting part 15b after molding. The runner F′ is branched from the sprue E and faces the insertion hole 30c. The gate G′ is an injection port of the resin P to the insertion hole 30c. As a result, the end part side of the exterior member 15 as illustrated in FIG. 4 to FIG. 6 is integrally formed.

Note that, when the end part side of the exterior member 15 into which the supporting member 30 has been inserted is removed from the mold, the sprue E and the runners F and F′ are detached from the exterior member 15, and only the gates G and G′ remain in the exterior member 15.

Further, the light guide pipe sleeve 10 is inserted into the light guide pipe sleeve insertion hole 30a of the supporting member 30, and the air feeding pipe sleeve 11 is inserted into the air feeding pipe sleeve insertion hole 30b of the supporting member 30.

At this time, the reason why the resin P is injected into the mold from the proximal end side of the first fitting part 15a in the fitting direction S is to prevent the remaining gates G and G′ from appearing on an outer surface such as the distal end surfaces 15af and 15bf on the end part side of the exterior member 15 and the outer peripheral surface, because the end part side of the exterior member 15 is an outside appearance member as illustrated in FIG. 1 and FIG. 2.

Note that the other configuration on the end part side of the endoscope connector 5 is the same as the existing configuration, and the description of the configuration is therefore omitted.

As mentioned above, in the present embodiment, in the supporting member 30, the insertion hole 30c that penetrates through the supporting member 30 in the fitting direction S so as to face the distal end surface 15bf and is filled with the filling part 15j integral with the distal end surface 15bf, is so provided as to face the region that is different from the second region B and the third region C. More specifically, the insertion hole 30c is so provided as to face the first region A as described above.

Accordingly, when the resin P is injected into the mold through the sprue E, the runners F and F′, and the gates G and G′ to form the end part side of the exterior member 15, the resin P flows from the first region A into a part that becomes the distal end surface 15bf, through the insertion hole 30c, in addition to the outer periphery on the end part side of the exterior member 15. This makes it possible to shift an occurrence position of the above-described weld line from the narrow first region A. For example, as illustrated in FIG. 4, it is possible to shift the occurrence position of the weld line to a region D having a large area on the distal end surface 15af.

Accordingly, strength on the end part side of the exterior member 15 is not deteriorated when the weld line occurs in the region D having the large area on the distal end surface 15af. This eliminates deterioration of chemical resistance on the end part side of the exterior member 15 due to the strength deterioration.

Therefore, it is possible to provide the endoscope connector 5 having the configuration that makes it possible to sufficiently secure the strength of the end part side even when the end part side of the exterior member 15 in the first fitting part 15a and the second fitting part 15b is formed integrally with the other part of the exterior member 15 in the first fitting part 15a and the second fitting part 15b.

Note that a modification is described below. In the above-described embodiment, the insertion hole 30c is so provided as to face the region that is different from the second region B and the third region C of the distal end surface 15bf, more specifically, as to face the first region A.

Furthermore, the insertion hole 30c may be so provided as to face, for example, one of a fourth region H and a fifth region I of the distal end surface 15bf as illustrated in FIG. 4 without being limited to the first region A as long as the region is different from the second region B and the third region C of the distal end surface 15bf.

This causes the resin P to flow into the part that becomes the distal end surface 15bf from the fourth region H or the fifth region I through the insertion hole 30c, in addition to the outer periphery of the end part side of the exterior member 15 when the resin P is injected into the mold through the sprue E, the runners F and F′, and the gates G and G′. Accordingly, the weld line occurs in a region shifted from the first region A, which makes it possible to produce effects similar to the effects of the present embodiment.

### Second Embodiment

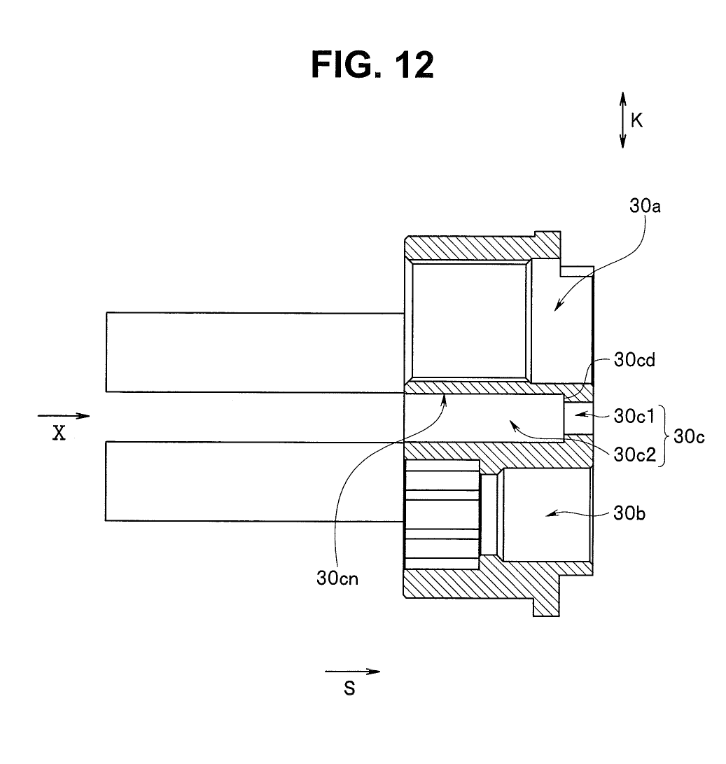

FIG. 12 is a cross-sectional diagram of an internal member in an endoscope connector according to the present embodiment.

A configuration of the endoscope connector according to the second embodiment is different from the configuration of the endoscope connector according to the above-described first embodiment illustrated in FIG. 1 to FIG. 11 in that a step part is provided in the insertion hole.

Therefore, only the difference is described, and components similar to the components of the first embodiment are denoted by the same reference numerals and description of such components is omitted.

As illustrated in FIG. 12, in the present embodiment, the insertion hole 30c is formed in a shape that increases a contact area between an inner peripheral surface 30cn and the exterior member filled in the insertion hole 30c, namely, the filling part 15j. More specifically, the insertion hole 30c is formed in an undercut shape having a step 30cd. The step 30cd is formed when a part 30c2 is formed so as to be larger in diameter than a part 30c1 on the distal end side in the fitting direction S.

This is because, if the insertion hole 30c does not have the step 30cd, the inner surface of the first opening 15h1 and the inner surface of the second opening 15h2 may stick to the mold when the end part side of the exterior member 15 is removed from the mold as described above.

In contrast, when the insertion hole 30c has the step 30cd, the filling part 15j formed integrally with the distal end surface 15bf is caught by the step 30cd when the end part side of the exterior member 15 is removed from the mold. This causes the inner surface of the first opening 15h1 and the inner surface of the second opening 15h2 to be easily detached from the mold without sticking to the mold. Note that the other configuration is the same as the configuration of the above-described first embodiment.

As mentioned above, in the present embodiment, the insertion hole 30c is formed in the undercut shape having the step 30cd.

Therefore, the filling part 15j formed integrally with the distal end surface 15bf is caught by the step 30cd when the end part side of the exterior member 15 is removed from the mold.

Accordingly, the inner surface of the first opening 15h1 and the inner surface of the second opening 15h2 are easily detached from the mold without sticking to the mold, which makes it possible to improve the strength of the exterior member 15. Note that other effects are the same as the effects of the above-described first embodiment.

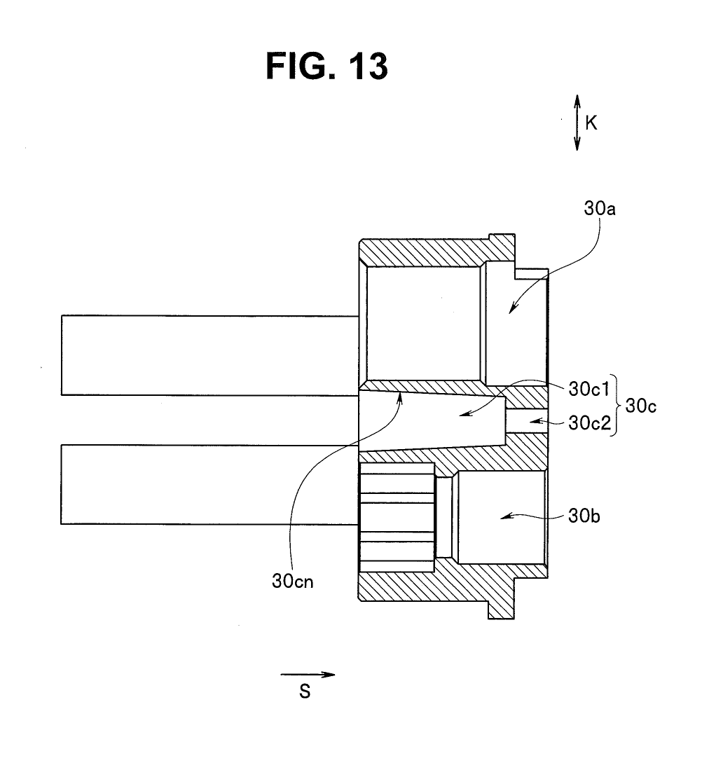

In addition, another modification is described below with reference to FIG. 13. FIG. 13 is a cross-sectional diagram illustrating a modification in which the inner peripheral surface of the insertion hole of the supporting member of FIG. 12 is formed in a tapered shape.

In the above-described embodiment, the insertion hole 30c is formed in the shape that increases the contact area between the inner peripheral surface 30cn and the filling part 15j filled in the insertion hole 30c. More specifically, the insertion hole 30c is formed in the undercut shape having the step 30cd.

Alternatively, the insertion hole 30c may be formed in a tapered shape as illustrated in FIG. 13 without limitation as long as the shape makes it possible to increase the contact area between the inner peripheral surface 30cn and the filling part 15j filled in the insertion hole 30c.

Note that, a cross-sectional surface of the tapered inner peripheral surface 30cn is not limited to a linear shape, and may be a shape provided with a plurality of steps although not illustrated.

Such a tapered inner peripheral surface 30cn makes it possible to produce effects similar to the effects by the shape of the inner peripheral surface 30cn having the step 30cd illustrated in FIG. 12, and the contact area between the tapered inner peripheral surface 30cn and the filling part 15j is increased as compared with the shape of the inner peripheral surface 30cn illustrated in FIG. 12.

Therefore, when the end part side of the exterior member 15 is removed from the mold as mentioned above, the filling part 15j formed integrally with the distal end surface 15bf is easily caught by the inner peripheral surface 30cn. This allows the end part side of the exterior member 15 to be easily removed from the mold without sticking of the distal end surface 15bf to the mold.

In addition, in the above-described first and second embodiments, the end part side of the exterior member 15 is formed in the two-stage shape having the first fitting part 15a and the second fitting part 15b.

The shape of the end part side of the exterior member 15 is not limited to the shape described above. A shape configured of only the first fitting part 15a or only the second fitting part 15b is adoptable as long as the first opening 15h1 and the second opening 15h2 are provided close to each other.

Further, in the above-described first and second embodiments, the light source apparatus 50 has been described as an example of the external apparatus. In other words, the case in which the connector receiver 20 to be attached with the endoscope connector 5 is provided in the light source apparatus 50 has been described as an example. Alternatively, the connector receiver 20 may be provided in the video processor 60 or other external apparatus without limitation.

In addition, in the above-described first and second embodiments, the first opening 15h1 and the second opening 15h2 of the exterior member 15 respectively expose the light guide pipe sleeve 10 and the air feeding pipe sleeve 11 as the predetermined members to the outside; however, the first opening 15h1 and the second opening 15h2 of the exterior member 15 may expose other predetermined members to the outside without limitation as a matter of course.

What is claimed is:

1. An endoscope connector for connecting an endoscope to an external apparatus, the endoscope connector comprising:
an exterior member including a first opening and a second opening on end part side, the first opening and the second opening respectively exposing, to an outside, predetermined members of the endoscope to be connected to the external apparatus; and
an internal member that is disposed inside the exterior member and holds the predetermined members, wherein
the internal member includes an insertion hole that faces a region on the end part side of the exterior member, the region being different from the first opening and the second opening.

2. The endoscope connector according to claim 1, wherein the insertion hole is filled with a portion of the exterior member.

3. The endoscope connector according to claim 2, wherein an end surface part located on the end part side of the exterior member has a filling part integral with the end surface part, the filling part projecting from the end surface part toward an inside of the insertion hole.

4. The endoscope connector according to claim 1, wherein
an end surface part located on the end part side of the exterior member includes a first region, a second region, and a third region, the first region being provided between the first opening and the second opening in a radial direction of the exterior member, the second region facing the first region with the first opening in between in the radial direction, and the third region facing the first region with the second opening in between in the radial direction, and
the insertion hole is provided to face a region on the end surface part, the region being different from the second region and the third region.

5. The endoscope connector according to claim 4, wherein the insertion hole is provided to face the first region on the end surface part.

6. The endoscope connector according to claim 2, wherein the insertion hole has an inner peripheral surface formed in a shape that increases a contact area between the inner peripheral surface and the exterior member filled in the insertion hole.

7. The endoscope connector according to claim 6, wherein the insertion hole has a step shape on the inner peripheral surface.
